SEQUENCE LISTING (20 sequences)

SEQ ID NO: 1 - DNA, 22 bp, Artificial Sequence
  Primers
atgaaaaaactcttgaaatcgg

SEQ ID NO: 2 - DNA, 26 bp, Artificial Sequence
  Primers
caagattttctaganttcatnttgtt

SEQ ID NO: 3 - DNA, 26 bp, Artificial Sequence
  Primers
caagcttttctagatttcatcttgtt

SEQ ID NO: 4 - DNA, 26 bp, Artificial Sequence
  Primers
caagcttttctagatttcattttgtt

SEQ ID NO: 5 - DNA, 26 bp, Artificial Sequence
  Primers
caagcttttctagacttcatcttgtt

SEQ ID NO: 6 - DNA, 26 bp, Artificial Sequence
  Primers
caagcttttctagacttcattttgtt

SEQ ID NO: 7 - DNA, 52 bp, Chlamydia pneumoniae
ggtttattcggagttaaaggtactactgtaaatgcaaatgaactaccaaacg

SEQ ID NO: 8 - DNA, 49 bp, Chlamydia pecorum
ggtttaattgggatttctgggtcaaccctgaatgataagctcccaaacg

SEQ ID NO: 9 - DNA, 52 bp, Chlamydia abortus
ggtttgattggtgttaaaggatcctccatagcagctgatcagcttcccaatg

SEQ ID NO: 10 - DNA, 63 bp, Chlamydia psittacci
ggttaatagggttttcagctgcaagctcaatctctaccgatcttccaatgcaacttcctaacg

SEQ ID NO: 11 - DNA, 56 bp, Chlamydia trachomatis
ggattgtttggagataatgaaaatcaaaaaacggtcaaagcggagtctgtaccaaa

SEQ ID NO: 12 - DNA, 22 bp, Artificial Sequence
  Primers
gctgatcagctgctatggagga

SEQ ID NO: 13 - DNA, 22 bp, Artificial Sequence
  Primers
ttgcattggaagatcggtagag

SEQ ID NO: 14 - DNA, 22 bp, Artificial Sequence
  Primers
cgtttggtagttcatttgcatt

SEQ ID NO: 15 - DNA, 22 bp, Artificial Sequence
  Primers
agcttatcattcagggttgacc

SEQ ID NO: 16 - DNA, 22 bp, Artificial Sequence
  Primers
tacagactccgctttgaccgtt

SEQ ID NO: 17 - DNA, 44 bp, Artificial Sequence
  based on C. pneumonia - C. abortus
cgtttggtagttcatttgcattgctgatcagctgctatggagga

SEQ ID NO: 18 - DNA, 44 bp, Artificial Sequence
  based on C. pneumonia - C. psittacci
cgtttggtagttcatttgcattttgcattggaagatcggtagag

SEQ ID NO: 19 - DNA, 44 bp, Artificial Sequence
  based on C. abortus -C. pneumonia
gctgatcagctgctatggaggacgtttggtagttcatttgcatt

SEQ ID NO: 20 - DNA, 44 bp, Artificial Sequence
  based on C. psittacci - C. pneumoniae
ttgcattggaagatcggtagagcgtttggtagttcatttgcatt

(19) United States
(12) Patent Application Publication
(10) Pub. No.: US 2004/0002074 A1
(43) Pub. Date: Jan. 1, 2004

(54) Hybrid oligonucleotide primers for amplification of DNA and uses thereof

(76) Inventors: Chalom Sayada, Luxembourg City (LU); Erick Denamur, Paris (FR); Gary Magnant, Topsfield, MA (US)

Correspondence Address: CLARK & ELBING LLP, 101 FEDERAL STREET, BOSTON, MA, 02110, US

(21) Appl. No.: 10/300,369

(22) Filed: Nov. 20, 2002

Related U.S. Application Data
(60) Provisional application No. 60/331,915, filed on Nov. 20, 2001.

(51) Int. Cl. C12Q 1/68; C07H 21/04; C12P 19/34

(52) U.S. Cl. 435/6; 536/23.7; 435/91.2

(57) ABSTRACT

The present invention provides compositions and methods for the determination of the presence of specific microorganisms in a sample using the amplification of DNA. The invention further provides compositions and methods that provide an internal standard for the validation of the amplification of DNA. The invention is based on the use of hybrid oligonucleotides that have two domains that are specific for two genetically distinct regions of DNA, e.g., in two genetically distinct microorganisms.

### CROSS-REFERENCE TO RELATED APPLICATIONS

The application claims benefit of U.S. Provisional Application No. 60/331,915, filed Nov. 20, 2001, hereby incorporated by reference.

### BACKGROUND OF THE INVENTION

The invention relates to the field of diagnostics for detecting and monitoring microbial infection.

Microbial infection is responsible for a wide variety of diseases, with different microorganisms acting as the causative agent in many of these diseases. Infections by these microorganisms sometimes cause similar symptoms, but effective treatments may differ depending on the nature of the causative organism. The ability to determine the specific microorganism responsible for an infection may therefore be important in order to treat a disease effectively and to prevent more serious conditions or death from occurring.

One method of positively identifying an organism is to test for the presence of specific genes that are only found in one species or strain of microorganism. This testing can be done using DNA amplification by, for example, the polymerase chain reaction (PCR). The method, however, requires primers that are specific for a region of DNA in only one species or strain of microorganism. In addition, the method is susceptible to false negative results. Thus, there is a need for improved methods for detecting the presence of microorganisms in a sample.

### SUMMARY OF THE INVENTION

The present invention provides compositions and methods for the determination of the presence of specific microorganisms in a sample using the amplification of DNA. The invention further provides compositions and methods that provide an internal standard for the validation of an amplified sample of DNA. The invention is based on the use of hybrid oligonucleotides that have two domains that are specific for two distinct regions of DNA, e.g., from two genetically distinct microorganisms. One of the domains (“the 3′ domain”) is disposed at the 3′ end of the oligonucleotide, and the other domain (“the 5′ domain”) is disposed 5′ to the 3′ domain.

In one aspect, the invention features a method of detecting a microorganism. The first step includes providing a pair of oligonucleotides and a DNA standard including a first region and a second region, wherein the pair includes a first oligonucleotide having a first domain that binds the first region of the DNA standard and is disposed at the 3′ end of the first oligonucleotide, and a second domain that binds a first region of DNA of a first type of microorganism and is disposed 5′ to the first domain; and a second oligonucleotide having a first domain that binds the second region of the DNA standard and a second domain that binds a second region of DNA of the first type of microorganism, wherein the first and second regions of DNA of the first type of microorganism flank a gene, or portion thereof, of the first type of microorganism, and wherein the first and second oligonucleotides are together capable of amplifying the DNA standard, or portion thereof, under appropriate conditions. The method further includes contacting the DNA standard and the pair of oligonucleotides with a first solution for a first amplification of DNA; and performing the first amplification of DNA in the first solution to produce a first amplification product. The first amplification product is then contacted with a second solution for a second amplification of DNA, wherein the second solution includes a sample, a first primer that binds the first region of DNA of the first type of microorganism, and a second primer that binds the second region of DNA of the first type of microorganism, wherein the first and second primers are together capable of amplifying DNA of the first amplification product and of the gene, or portion thereof, of the first type of microorganism under appropriate conditions; and the second amplification is performed in the second solution to produce a second amplification product. The second amplification product is then analyzed for the presence of amplified DNA from the first type of microorganism and the DNA standard, wherein the presence of DNA from the first type of microorganism and the DNA standard is indicative of the presence of the first type of microorganism in the sample; the absence of DNA from the first type of microorganism and the presence of DNA from the DNA standard is indicative of the absence of the first type of microorganism in the sample; and the absence of DNA from the first type of microorganism and the DNA standard is indicative of a failure in the first or second amplification.

Desirably, the DNA standard includes a gene, or portion thereof, of a second type of microorganism, and the first and second regions of the DNA standard flank that gene, or portion thereof. In another embodiment, the second type of microorganism is the same as the first type of microorganism. The first domain may also be the second domain in the second oligonucleotide, or the second domain of the second oligonucleotide may be disposed 5′ to the first domain. In various embodiments, the first and second primers are not capable of amplifying DNA from another microorganism potentially present in the sample.

Alternatively, the DNA standard may be an artificial sequence, e.g., one that has a length similar to that of the DNA flanked by the primers in the second amplification. In various embodiments, amplifications of DNA employed in the above methods are amplifications by the polymerase chain reaction (PCR), the sample is a biological sample, and the methods include the additional step of isolating the amplified DNA from the first amplification before using it as an internal standard in the second amplification.

The invention further features a pair of hybrid oligonucleotides including a first oligonucleotide having a first domain that binds to a first region of DNA of a first type of microorganism and is disposed at the 3′ end of the first oligonucleotide, and a second domain that binds to a first region of DNA of a second type of microorganism and is disposed 5′ to the first domain of the first oligonucleotide; and a second oligonucleotide having a first domain that binds to a second region of DNA of the first type of microorganism and is disposed at the 3′ end of the second oligonucleotide, and a second domain that binds to a second region of DNA of the second type of microorganism and is disposed 5′ to the first domain, wherein the first and second regions of DNA of the first type of microorganism flank a gene, or portion thereof, of the first type of microorganism, and the first and second regions of DNA of the second type of microorganism flank a gene, or portion thereof, of the second type of microorganism, and wherein the first and second oligonucleotides are together capable of amplifying DNA of the gene of the first type of microorganism, or portion thereof, under appropriate conditions. In one embodiment, the two genes to which the 3′ and 5′ domains bind are orthologous genes in different microorganisms. In another embodiment, the two genes are different genes in the same microorganism. In yet another embodiment, the pair are used as primers for the amplification of the DNA that the 3′ domains flank.

In another aspect, the invention features a hybrid oligonucleotide having a first domain that binds to a region of DNA of a first type of microorganism and is disposed at the 3′ end of the oligonucleotide, and a second domain that binds to a region of DNA of a second type of microorganism and is disposed 5′ to the first domain. Exemplary hybrid oligonucleotides are described herein.

In particular embodiments of any of the above aspects, the microorganisms are bacteria. Suitable bacteria are listed below. Preferably, the bacteria are members of the order Chlamydiales (e.g., Chlamydia (C.) pneumoniae, C. psittacci, C. abortus, C. trachomatis, Simkania negevensis, and Parachlamydia acanthamoebae). Desirably, the gene that the oligonucleotides or primers of the invention flank is the gene for the major-outer-membrane protein.

The methods and oligonucleotides of the invention may also be adapted for use for the amplification of nucleic acids from organisms other than microorganisms or on completely artificial sequences of DNA.

By “microorganism” is meant an organism of microscopic size including, without limitation, bacteria, fungi, algae, protozoa, and viruses.

By “type of microorganism” is meant a species or strain of microorganism.

By a sequence of nucleotides “disposed at the 3′ end” is meant one that includes the 3′ end of the oligonucleotide.

By a sequence of nucleotides “disposed 5′ to a domain” is meant a sequence covalently bound directly to the 5′ end of the domain or covalently bound to the 5′ end of another sequence that is itself covalently bound to the 5′ end of the domain.

By “domain” is meant a sequence of 10 or more nucleotides that binds to a second sequence of nucleotides (or region) in a molecule of DNA, under conditions suitable for the amplification of DNA.

By “hybrid oligonucleotide” is meant an oligonucleotide that includes two or more domains, wherein the nucleic acid sequences that the domains specifically bind to do not naturally occur adjacent to one another. A hybrid oligonucleotide may, for example, have at least 20, 30, 40, 50, 60, 70, 80, 90, or more than 90 nucleotides, or at most 90, 80, 70, 60, 50, 40, or 30 nucleotides.

By “non-hybrid oligonucleotide” is meant an oligonucleotide that does not include two or more domains that do not naturally occur adjacent to one another.

By “primer” is meant an oligonucleotide that binds to a specific sequence of nucleic acid and can be elongated by a polymerase under appropriate conditions. A set of two primers is used to amplify the sequence of DNA flanked by the primers. Primers may also be general or genus-, species-, or strain-specific. A primer may contain commonly occurring bases adenine, cytosine, guanine, thymine, or uracil, and modified bases, such as inosine.

The term “microbial infection” refers to the invasion of the host mammal by pathogenic microorganisms (e.g., bacteria, fungi, yeasts, viruses, or protozoa). This term includes the excessive growth of microorganisms that are normally present in or on the body of a mammal. More generally, a microbial infection can be any situation in which the presence of a microbial population(s) is damaging to a host mammal. Thus, a mammal is “suffering” from a microbial infection when excessive numbers of a microbial population are present in or on a mammal's body, or when the presence of a microbial population(s) is damaging the cells or other tissue of a mammal.

By “orthologous” is meant evolved from the same parent gene after speciation.

### BRIEF DESCRIPTION OF THE DRAWINGS

FIG. 1A is a schematic view of a hybrid oligonucleotide, including a 3′ domain and a 5′ domain.

FIG. 1B is a schematic view of an amplification of DNA using a non-hybrid oligonucleotide and a hybrid oligonucleotide as primers.

FIG. 1C is a schematic view of an amplification of DNA in a sample using DNA amplified in FIG. 1B as an internal standard. The non-hybrid oligonucleotide from FIG. 1B and a non-hybrid oligonucleotide, capable of binding to the complement of the 5′ domain in FIG. 1A and amplifying DNA, are used as primers.

### DETAILED DESCRIPTION OF THE INVENTION

The invention features hybrid oligonucleotide primers. These primers include two domains, a 3′ (or first) domain and a 5′ (or second) domain, each capable of being used separately as a primer for nucleic acid amplification. The domains may be primers that are specific for a gene or region of a gene of a specific species or strain of microorganism (e.g., a bacterium). The domains may also be primers for different genes, or different portions of the same gene, in the same microorganism. The structure of the hybrid primer is such that it can only be used to amplify nucleic acid to which the 3′ domain will bind, and its use results in an amplification product that contains nucleic acid sequences from the 5′ domain of the primer. This product can then be used as an internal standard when amplifying nucleic acid using a primer based on the 5′ domain of the hybrid primer.

The invention also features methods for diagnosing the presence of a microorganism (e.g., a bacterium) in a biological material, as well as to the use of these methods to evaluate the serological status of an individual undergoing antimicrobial therapy. For purposes of this application, “biological material” includes, but is not limited to, bodily secretions, bodily fluids, and tissue specimens. Examples of bodily secretions include cervical secretions, trachial-bronchial secretions, and pharyngeal secretions. Suitable bodily fluids include, without limitation, blood, sweat, tears, cerebral spinal system fluid, serum, sputum, earwax, urine, synovial fluid, and saliva. Animals, cells, and tissue specimens such as from a variety of biopsies are also embraced by this term.

In certain embodiments, the microbial infection to be detected by the invention is an infection of a bacterium selected from the group consisting of Chlamydia pneumoniae, C. psittacci, C. abortus, C. trachomatis, or other member of the order Chlamydiales (e.g., Simkania negevensis, Parachlamydia acanthamoebae), Pseudomonas aeruginosa, P. alcaligenes, P. chlororaphis, P. fluorescens, P. luteola, P. mendocina, P. monteilii, P. oryzihabitans, P. pertocinogena, P. pseudalcaligenes, P. putida, P. stutzeri, Burkholderia cepacia, Aeromonas hydrophilia, Escherichia coli, Citrobacter freundii, Salmonella typhimurium, S. typhi, S. paratyphi, S. enteritidis, Shigella dysenteriae, S. flexneri, S. sonnei, Enterobacter cloacae, E. aerogenes, Klebsiella pneumoniae, K. oxytoca, Serratia marcescens, Francisella tularensis, Morganella morganii, Proteus mirabilis, Proteus vulgaris, Providencia alcalifaciens, P. rettgeri, P. stuartii, Acinetobacter calcoaceticus, A. haemolyticus, Yersinia enterocolitica, Y. pestis, Y. pseudotuberculosis, Y. intermedia, Bordetella pertussis, B. parapertussis, B. bronchiseptica, Haemophilus influenzae, H. parainfluenzae, H. haemolyticus, H. parahaemolyticus, H. ducreyi, Pasteurella multocida, P. haemolytica, Branhamella catarrhalis, Helicobacter pylori, Campylobacter fetus, C. jejuni, C. coli, Borrelia burgdorferi, V. cholerae, V. parahaemolyticus, Legionella pneumophila, Listeria monocytogenes, Neisseria gonorrhea, N. meningitidis, Kingella dentrificans, K. kingae, K. oralis, Moraxella catarrhalis, M. atlantae, M. lacunata, M. nonliquefaciens, M. osloensis, M. phenylpyruvica, Gardnerella vaginalis, Bacteroides fragilis, Bacteroides distasonis, Bacteroides 3452A homology group, Bacteroides vulgatus, B. ovalus, B. thetaiotaomicron, B. uniformis, B. eggerthii, B. splanchnicus, Mycobacterium tuberculosis, M. avium, M. intracellulare, M. leprae, Clostridium difficile, C. diphtheriae, C. ulcerans, C. accolens, C. afermentans, C. amycolatum, C. argentorense, C. auris, C. bovis, C. confusum, C. coyleae, C. durum, C. falsenii, C. glucuronolyticum, C. imitans, C. jeikeium, C. kutscheri, C. kroppenstedtii, C. lipophilum, C. macginleyi, C. matruchoti, C. mucifaciens, C. pilosum, C. propinquum, C. renale, C. riegelii, C. sanguinis, C. singulare, C. striatum, C. sundsvallense, C. thomssenii, C. urealyticum, C. xerosis, Streptococcus pneumoniae, S. agalactiae, S. pyogenes, Enterococcus avium, E. casseliflavus, E. cecorum, E. dispar, E. durans, E. faecalis, E. faecium, E. flavescens, E. gallinarum, E. hirae, E. malodoratus, E. mundtii, E. pseudoavium, E. raffinosus, E. solitarius, Staphylococcus aureus, S. epidermidis, S. saprophyticus, S. intermedius, S. hyicus, S. haemolyticus, S. hominis, and S. saccharolyticus. Accordingly, the invention discloses a method of detecting infections by the bacteria above, among others.

Any known primer-based technique for nucleic acid (e.g., DNA and RNA) amplification can be used with the assays described herein. Suitable amplification techniques include the polymerase chain reaction (PCR) methodologies, which include solution PCR and in situ PCR, for detection of the presence or absence of unique genes of microorganisms.

The nucleotide sequence of the primers used in the amplification of DNA determines the specificity of the method, e.g., for a particular gene and/or a particular species or strain of bacteria. The use of primers that bind to the same gene for a group of different species or strains provides a tool for the detection of a microorganism, e.g., a bacterium, in that group. In some instances, the identification of the group is sufficient to determine a course of treatment. Table 1 shows examples of primers that bind to more than one species of bacteria.

TABLE 1

Primers for the amplification of the MOMP gene of
the order Chlamydiales.

| | | SEQ |
|---|---|---|
| Primer | | ID |
| ID | Primer sequence | No. |
| CTU | 5′ATGAAAAAACTCTTGAAATCGG-3′ | 1 |
| CTL | 5′CAAGATTTTCTAGA(T/C)TTCAT(C/T)TTGTT3′ | 2 |
| CTL1 | 5′CAAGCTTTTCTAGATTTCATCTTGTT3′ | 3 |
| CTL2 | 5′CAAGCTTTTCTAGATTTCATTTTGTT3′ | 4 |
| CTL3 | 5′CAAGCTTTTCTAGACTTCATCTTGTT3′ | 5 |
| CTL4 | 5′CAAGCTTTTCTAGACTTCATTTTGTT3′ | 6 |

In some instances, effective treatment of a microbial infection requires information on the precise nature of the microorganism. In such instances, assays that are specific for species or strains can be used to further identify the microorganism. Species- or strain-specific assays for detecting microorganisms can be designed based upon the primers selected.

In one example, GeneBank available sequences of the major-outer-membrane protein (MOMP) region of different Chlamydia species are aligned using the CLUSTAL method, and the region from nucleotide 466 to 523 is selected for species-specific primer design because it shows high divergence among the different species. The sequence alignment of this region is shown in Table 2. Underlined nucleotides represent the sequences used for primer design.

TABLE 2

Nucleic acid sequences of the MOMP gene for species of Chlamydia.

| | | |
|---|---|---|
| | | SEQ |
| | | ID |
| Species | Sequence | No. |

| | | |
|---|---|---|
| C. pneumoniae | 5′ggtttattcggagttaaaggt------------actactgtaaatgcaaatgaactaccaaacg 3′ | 7 |
| C. pecorum | 5′ ggtttaattgggatttctggg------------tcaaccctgaatg----ataagctcccaaacg 3′ | 8 |
| C. abortus | 5′ ggtttgattggtgttaaagga-------------tcctccatagcagctgatcagcttcccaatg 3′ | 9 |
| C. psittacci | 5′ ggttaatagggttttcagctgcaagctcaatctctaccgatcttccaatgcaacttcctaaqcg 3′ | 10 |
| C. trachomatis | 5′ ggattgtttggagataatgaaaatcaaaaa------acggtcaaagcggagtctgtaccaaa 3′ | 11 |

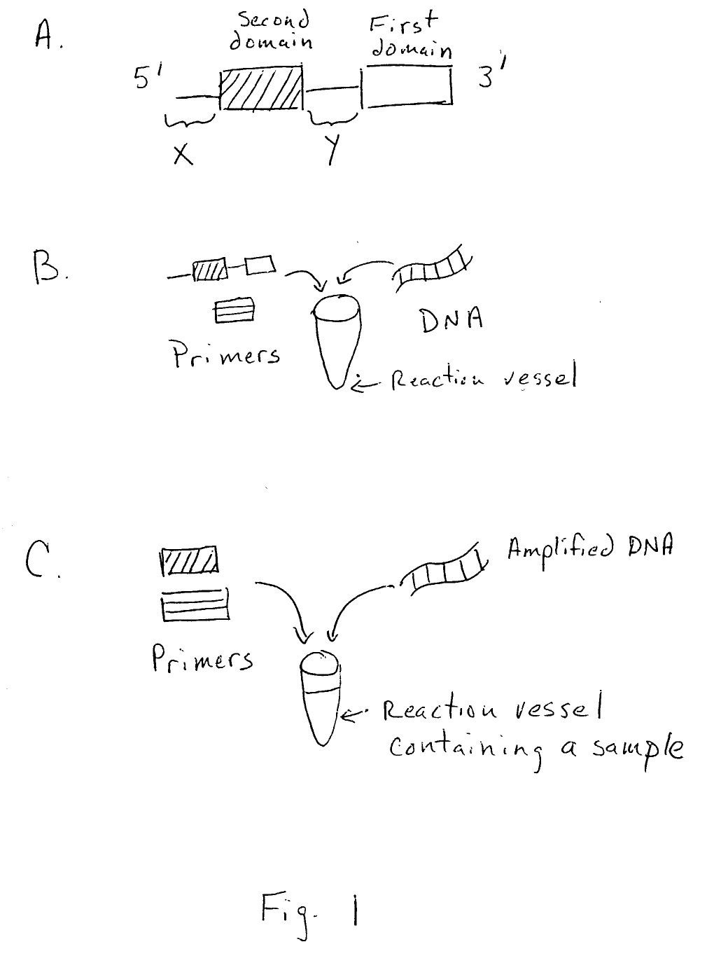

Table 3 shows examples of primers that are specific for species of Chlamydia when used in combination with the CTU primer. Hybrid primers can also be used in an amplification assay to determine the presence of a specific bacterium. A schematic example of a hybrid oligonucleotide is shown in FIG. 1A. The first (or 3′) domain is located at the 3′ end of the oligonucleotide. The second (or 5′) domain is located 5′ to the first domain. In one embodiment, a sequence Y separates the two domains, and a sequence X is located 5′ to the second domain. The sequences X and Y may also be absent. The hybrid primers of this invention are used in pairs or in combination with primers that bind to more than one species or strain in the assays. Examples of desired hybrid primers are given in Table 4. The 3′ domains of the hybrid primers are underlined.

TABLE 3

Primers for the amplification of the MOMP gene of specific
species of Chlamydiales.

| | | | | | |
|---|---|---|---|---|---|
| | | Starting | Length | | SEQ |
| | | primer | of PCR | | ID |
| Primer ID | Primer sequence | (5′)* | product | Source | No. |

| | | | | | |
|---|---|---|---|---|---|
| CTU | 5′ATGAAAAAACTCTTGAAATCGG-3′ | 1 | | | 1 |
| L abortus | 5′GCTGATCAGCTGCTATGGAGGA-3′ | 487 | 508 | strain B577 | 12 |
| | | | (accession no. | | |
| | | | M73036) | | |
| L psittacci | 5′TTGCATTGGAAGATCGGTAGAG-3′ | 510 | 531 | strain 6BC | 13 |
| | | | (accession no. | | |
| | | | M73035) | | |
| L pneumoniae | 5′CGTTTGGTAGTTCATTTGCATT-3′ | 496 | 517 | strain AR39 | 14 |
| | | | (accession no. | | |
| | | | M73035) | | |
| L pecorum | 5′AGCTTATCATTCAGGGTTGACC-3′ | 488 | 509 | strain 66P130 | 15 |
| | | | (accession no. | | |
| | | | M73034) | | |
| L trachomatis | 5′TACAGACTCCGCTTTGACCGTT-3′ | 498 | 519 | strain D/LSU- | 16 |
| | | | PM12 | | |
| | | | (accession no. | | |
| | | | | AF279588) | |

*Start position of primer relative to the first nucleotide of the starting codon of the gene.

PCR techniques are discussed in detail below in the Example Section. In general, solution PCR is carried out on nucleic acid isolated from a biological material. For example, in species of Chlamydia, biological material is first pre-incubated in an appropriate reducing agent that is capable of reducing the disulfide bonds that maintain the integrity of the MOMP and other surface proteins of the chlamydial elementary bodies (EBs), thereby compromising the outer protective shell of the EBs and allowing protease penetration. Suitable disulfide reducing agents include, but are not limited to, dithiothreitol, succimer, glutathione, DL-penicillamine, D-penicillamine disulfide, 2,2′-dimercaptoadipic acid, and 2,3-dimercapto-1-propone-sulfide acid. Appropriate concentrations of these reducing agents are readily determined by the skilled artisan without undue experimentation using a 10-μM concentration of dithiothreitol (the desired reducing agent) as a guideline. Failure to include a reducing agent in the initial step may prevent DNA of EBs from being isolated in the subsequent step. In vitro data shows that dithiothreitol is most effective at opening EBs for protease digestion. In an alternative method, guanidine thiocyanate, at a concentration of 4 M, or functionally equivalent reducing denaturant, is substituted for the disulfide reduction/protease steps.

TABLE 4

Hybrid primers for the amplification of the MOMP gene of the order
Chlamydiales.

| | | SEQ |
|---|---|---|
| | | ID |
| Species | Hybrid Primer | No. |
| C pneumoniae- | pn:abor-R | 17 |
| C. abortus | 5′CGTTTGGTAGTTCATTTGCATTGCTGATCAGCTGCTATGGAGGA 3′ | |
| C. pneumoniae- | pn:psitt-R | 18 |
| C. psittacci | 5′CGTTTGGTAGTTCATTTGCATTTTGCATTGGAAGATCGGTAGAG 3′ | |
| C abortus-C. | abor:pn-R | 19 |
| C. pneumoniae | 5′GCTGATCAGCTGCTATGGAGGACGTTTGGTAGTTCATTTGCATT 3′ | |
| C psittacci- | psitt:pn-R | 20 |
| C. pneumoniae | 5′TTGCATTGGAAGATCGGTAGAGCGTTTGGTAGTTCATTTGCATT 3′ | |

The appropriate conditions for amplification of DNA using hybrid oligonucleotides or other primers of the invention will depend on the sequence and length of the primer and the number of species or strains that are to be detected (i.e., the desired level of specificity). One skilled in the art can determine suitable times, temperatures, enzymes, primer concentrations, and salt concentrations for annealing, extension, and denaturation (see, for example, Ausubel et al. Current Protocols in Molecular Biology Wiley: New York 1999). In general, higher stringency conditions lead to the amplification of fewer DNA sequences. This feature may be desirable, for example, when the amplification of DNA from one strain that is closely related to other strains (i.e., the DNA of the strains has a high degree of sequence homology, e.g., 90, 95, or <99%) is desired. Alternatively, lower stringency conditions may be used, for example, when the amplification of DNA from one of several closely related strains is desired.

Once the outer shell of the EBs has been released, the pre-incubated material is subjected to protein digestion using a protease (e.g., proteinase K), or functionally equivalent enzyme. The DNA is extracted and subjected to a nucleic acid amplification technique, e.g., PCR. The entire gene or portion thereof containing unique antigenic determinant(s) encoding MOMP or another suitable gene is amplified using appropriate primers flanking the gene to be amplified. For example, the gene or portion thereof can be the gene encoding MOMP, OMP-B, GRO-ES, GRO-EL, DNAK, 16S RNA, 23S RNA, the gene encoding ribonuclease-P, a 76 kDa attachment protein, or a KDO-transferase gene.

The amplified DNA may then be separated and identified by standard electrophoretic techniques. DNA bands may be identified using ethidium bromide staining and UV light detection. PCR primers may be designed to amplify DNA encoding MOMP of a particular Chlamydia species, such as the MOMP of C. pneumoniae, C. pecorum, C. trachomatis, C. psittacci, selectively. Primers that are from about a 15-mer to about a 40-mer may be designed for this purpose.

Hybrid oligonucleotides are used to provide an internal standard for the detection of a microorganism. In one example, the use of hybrid primers produces amplification products of genes from a bacterium that the first domain is specific for (denoted 3′ bacterium). The amplification products contain sequences of DNA not normally found in that bacterium. These hybrid amplification products are used as internal standards for assays for a bacterium for which the second domain is specific (denoted 5′ bacterium). In this example, DNA of the 3′ bacterium is amplified using at least one hybrid oligonucleotide as a primer (FIG. 1B). The second primer is, e.g., a primer that binds to regions of DNA of more than one species or strain or a second hybrid oligonucleotide. Hybrid amplified DNA of the 3′ bacterium is then added to a sample to be tested for the 5′ bacterium (FIG. 1C). Primers matching the 5′ domains of the hybrid primers are used to amplify both the amplified DNA of the 3′ bacterium and any DNA present of the 5′ bacterium. Analysis of the product should yield amplified DNA of the hybrid DNA and DNA of the 5′ bacterium, if the 5′ bacterium is present in the sample. If no DNA is detected following amplification, then a failure in the amplification method has occurred. In an alternative embodiment, the 3′ domain is used to amplify artificial DNA, which is then used as the internal standard. Prior to the second amplification, the internal standard DNA may be purified, e.g., to 80, 90, 95 or >95% purity, using techniques known in the art. The use of the hybrid primer allows for the same primers to be used to test for the presence of a bacterium and to validate the method. These primers thus provide a stringent test for the validation of the amplification.

Differentiating between the DNA of the internal standard and the sample requires a suitable technique for detection. Examples of techniques include hybridization assays; Southern blotting; electrophoresis of the intact DNA, provided there is a size difference between the two types of DNA; electrophoresis of digests of the DNA by restriction endonucleases provided there is a difference in size among the restriction fragments of the two types of DNA; and other techniques known by those skilled in the art.

The nucleic acid amplification techniques described above may be used to evaluate the course of antimicrobial therapy, e.g., antibacterial therapy. For example, the continued absence of detectable bacterial DNA encoding a specific gene as a function of antibacterial therapy is indicative of clinical management of the infection.

### EXAMPLE 1

### Amplification Of DNA By PCR

DNA amplifications were performed in a total volume of 25 μL containing 5 μL of DNA (1 ng is a sufficient quantity in order to obtain a strong signal), 2.5 μL of 10× PCR buffer for a 1× X final concentration, 1.5 mM of MgCl₂ (GibcoBRL®), each dATP, dCTP, dGTP, dTTP (GibcoBRL®) at 1.25 μM, 30 pmol of each primer, and 0.5 units of AMPLITAQ® DNA polymerase (GibcoBRL®).

Samples were subjected to 30 cycles of amplification in a DNA thermal cycler. Before cycling, samples were subjected to an initial step of denaturing for 5 min at 94° C. Cycling conditions were as follows: denaturation, 1 min at 94° C.; primer annealing, 1 min at 48° C.; and primer extension, 2 min at 72° C. After the 30 cycles, samples were submitted to a final step of extension, 7 min at 72° C., before finishing PCR amplification at 4° C. One negative control was systematically included in each series.

### EXAMPLE 2

### Amplification Of DNA Of Bacteria Using Primers Specific For More Than One Strain

DNA samples of strains of Chlamydia were amplified according to the method of Example 1. The primers used were those found in Table 1. Results of one experiment (shown in Table 5) indicate the primer's specificity to amplify the MOMP gene for Chlamydia strains.

TABLE 5
PCR amplification of Chlamydia with CTU/CTLn primers.

| | | | CTU/ | CTU/ | CTU/ | CTU/ | CTU/ |
|---|---|---|---|---|---|---|---|
| Microorganism | | Strains | CTL | CTL1 | CTL2 | CTL3 | CTL4 |
| C. abortus | 1 | B577 | + | + | + | + | + |
| | 2 | A-22 | + | + | + | + | + |
| | 3 | WT parakeet | + | + | + | + | |
| C. psittacci | 4 | 6-BC | + | + | + | + | |
| | 5 | CP-3 | (+) | − | − | + | + |
| | 6 | NJ−i | − | − | − | + | + |
| C. caviae | 7 | GPIC | + | + | + | + | + |
| C. felis | 8 | FEPN SCH | − | + | + | + | − |
| | | 56174 | | | | | |
| | 9 | FEPN PRING | − | − | − | − | − |
| | 10 | FEPN | + | + | + | + | + |
| | 11 | FEPN Baker | (+) | + | (+) | + | + |
| C. pneumoniae | 12 | Cdc/CWL-029 | ND | + | ND | ND | ND |
| | 13 | TW 183 1 ul | − | − | − | − | − |
| | 14 | TW 183 10 ul | − | − | − | − | − |
| | 15 | IOL-207 J1 | − | − | − | − | − |
| | 16 | IOL-207 J10 | − | − | − | − | − |
| C. pecorum | 17 | LW 679 | − | − | − | − | − |
| | 18 | Tc Sta | − | (+) | − | (+) | − |
| | 19 | 1710S | − | − | − | − | − |
| | 20 | L71 | − | − | − | (+) | − |
| | 21 | LW613 | − | + | + | + | (+) |
| | 22 | LW 623 | − | − | − | − | − |
| | 23 | IP1751 | − | − | − | − | − |
| | 24 | E-58 | − | + | − | + | − |
| C. trachomatis | 25 | Ref A | − | + | + | + | (+) |
| | 26 | Ref Ba | ND | + | + | + | + |
| | 27 | Ref G | + | + | + | + | + |
| | 28 | LGV2 | + | + | + | + | + |
| C. suis | 29 | L1 (porcine) | − | − | − | + | − |
| | 30 | S45 | + | + | + | + | + |
| C. muridarum | 31 | MOPN | − | + | + | + | − |
| S. negevensis | 32 | Z | ND | ND | ND | ND | ND |
| P. | 33 | Bn9 | − | − | ND | ND | ND |
| acanthamoebae | | | | | | | |

+: Amplification;
(+): Amplification not very strong;
−: No amplification;
ND: Strains not tested because of fewest DNA quantities

### EXAMPLE 3

### Amplification Of DNA Of Species Of Chlamydia Using Species-Specific Primers

DNA samples of strains of Chlamydia were amplified by the method of Example 1. The primers used were the species-specific primers of Table 3. Table 6 shows the results of the amplification. The results indicate the specificity of the species-specific primers to amplify the MOMP gene for specific strains.

TABLE 6
PCR amplification of the MOMP gene of Chlamydia using
species-specific primers.

| | | | CTU with specific primers | | | |
|---|---|---|---|---|---|---|
| | | | | CTU/ | | Control |
| | | | CTU/ | L | CTU/ | CTU/ |
| | | Strains | L abortus | psittacci | L pneumoniae | CTL3 |
| C. abortus | 1 | B577 | + | − | − | + |
| | 2 | A-22 | + | − | − | + |
| | 3 | WT parakeet | + | − | − | + |
| C. psittacci | 4 | 6-BC | − | + | − | + |
| | 5 | CP-3 | − | + | − | + |
| | 6 | NJ-1 | − | − | − | + |
| C. caviae | 7 | GPIC | − | − | − | + |
| C. felis | 8 | FEPN | − | − | − | + |
| C. pneumoniae | 9 | Cdc/CWL-029 | − | − | + | + |
| | 10 | J138 | ND | ND | + | ND |
| | 11 | Vanderbilt | − | − | − | − |
| C. pecorum | 12 | 66P130 | − | − | − | + |
| C. trachomatis | 13 | Ref A | − | − | − | + |
| | 14 | Ref G | − | − | − | + |
| | 15 | LGV2 | − | − | − | + |
| C. suis | 16 | S45 | − | − | − | + |
| C. muridarum | 17 | MOPN | − | − | − | + |

+: Amplification;
−: No amplification;
ND: Strains not tested because of fewest DNA quantities

### EXAMPLE 4

### Amplification Of DNA Of Species Of Chlamydia Using CTU And Hybrid Primers

DNA samples of strains of Chlamydia were amplified according to the method of Example 1. The primers used were those of Table 4 and CTU. The results are shown in Table 7. The results indicate that the hybrid primers only amplified the DNA of the strains to which the 3′ domains bound.

TABLE 7
PCR amplification of stains of Chlamydia using
hybrid primers and CTU.

| | | | CTU/ | CTU/ | CTU/ | CTU/ |
|---|---|---|---|---|---|---|
| | | Strains | abor:pn-R | psitt:pn-R | pn:abor-R | pn:psitt-R |
| C. abortus | 1 | B577 | − | − | + | − |
| | 2 | A-22 | − | − | + | − |
| | 3 | WT parakeet | − | − | + | − |
| C. psittacci | 4 | 6-BC | − | − | − | + |
| | 5 | CP-3 | − | − | − | + |
| | 6 | NJ-1 | − | − | − | − |
| C. caviae | 7 | GPIC | − | − | − | − |
| C. felis | 8 | FEPN | − | − | − | − |
| C. pneumoniae | 9 | Cdc/CWL-029 | + | + | − | − |
| | 10 | J138 | ND | ND | ND | ND |
| | 11 | Vanderbilt | ND | ND | ND | ND |
| C. pecorum | 12 | 66P130 | − | − | − | − |
| C. trachomatis | 13 | Ref A | − | − | − | − |
| | 14 | Ref G | − | − | − | − |
| | 15 | LGV2 | − | − | − | − |
| C. suis | 16 | S45 | − | − | − | − |
| C. muridarum | 17 | MOPN | − | − | − | − |

+: Amplification;
−: No amplification;
ND: Strains not tested because of fewest DNA quantities

### EXAMPLE 5

### Amplification Of DNA Of Species Of Chlamydia With An Internal Standard Using Pairs Of Hybrid Primers

DNA samples of strains of Chlamydia are amplified according to the method of Example 1. The primers used are pn:abor-R of Table 4 and another hybrid primer designed to replace the CTU primer with species-specific 3′ and 5′ domains. The amplified DNA of the MOMP gene of C. abortus contains known sequences of DNA from C. pneumoniae. The amplified DNA is added to a sample that is tested for the presence of C. pneumoniae. PCR amplification is performed on the sample according to the method of Example 1. The primers used are CTU and L pneumoniae of Table 3. The product of the amplification is analyzed for the presence of DNA. If DNA of C. abortus and C. pneumoniae is present, the sample contains C. pneumoniae. If DNA of C. abortus is present, but DNA of C. pneumoniae is absent, the sample does not contain C. pneumoniae. If no DNA is present, the amplification method failed, and the sample may or may not contain C. pneumoniae.

### EXAMPLE 6

### Amplification Of DNA Of Species Of Chlamydia With An Internal Standard Using CTU And A Hybrid Primer

DNA of the MOMP gene of C. abortus is amplified according to the method of Example 1. The primers used are CTU and pn:abor-R of Table 4. The amplified DNA is then isolated using standard methods. The amplified DNA of the MOMP gene of C. abortus contains a known sequence of DNA from C. pneumoniae. The amplified DNA is added to a sample that is tested for the presence of C. pneumoniae. PCR amplification is performed on the sample according to the method of Example 1. The primers used are CTU and L pneumoniae of Table 3. The product of the amplification is analyzed for the presence of DNA. If DNA of C. abortus and C. pneumoniae is present, the sample contains C. pneumoniae. If DNA of C. abortus is present, but DNA of C. pneumoniae is absent, the sample does not contain C. pneumoniae. If no DNA is present, the amplification method failed, and the sample may or may not contain C. pneumoniae.

### Other Embodiments

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

All patents, patent applications, and publications mentioned in this application are hereby incorporated by reference.

Other embodiments are within the claims.

What is claimed is:

1. A method of detecting a microorganism, said method comprising the steps of:
(a) providing a pair of oligonucleotides and a DNA standard comprising a first region and a second region, wherein said pair comprises:
(i) a first oligonucleotide comprising a first domain that binds said first region of said DNA standard and is disposed at the 3′ end of said first oligonucleotide, and a second domain that binds a first region of DNA of a first type of microorganism and is disposed 5′ to said first domain and
(ii) a second oligonucleotide comprising a first domain that binds said second region of said DNA standard and a second domain that binds a second region of DNA of said first type of microorganism,
wherein said first and second regions of DNA of said first type of microorganism flank a gene, or portion thereof, of said first type of microorganism, and wherein said first and second oligonucleotides are together capable of amplifying said DNA standard, or portion thereof, under appropriate conditions;
(b) contacting said DNA standard and said pair of oligonucleotides with a first solution for a first amplification of DNA;
(c) performing said first amplification of DNA in said first solution to produce a first amplification product; and
(d) contacting said first amplification product with a second solution for a second amplification of DNA, wherein said second solution comprises a sample, a first primer that binds said first region of DNA of said first type of microorganism, and a second primer that binds said second region of DNA of said first type of microorganism, wherein said first and second primers are together capable of amplifying DNA of said first amplification product and of said gene, or portion thereof, of said first type of microorganism under appropriate conditions;
(e) performing said second amplification in said second solution to produce a second amplification product; and
(f) analyzing said second amplification product for the presence of amplified DNA from said first type of microorganism and said DNA standard, wherein the presence of DNA from said first type of microorganism and said DNA standard is indicative of the presence of said first type of microorganism in said sample; the absence of DNA from said first type of microorganism and the presence of DNA from said DNA standard is indicative of the absence of said first type of microorganism in said sample; and the absence of DNA from said first type of microorganism and said DNA standard is indicative of a failure in said first or second amplification.

2. The method of claim 1, wherein said DNA standard comprises a gene, or portion thereof, of a second type of microorganism, and said first and second regions of said DNA standard flank said gene, or portion thereof, of said second type of microorganism.

3. The method of claim 2, wherein said second type of microorganism is said first type of microorganism.

4. The method of claim 1, wherein in said second oligonucleotide said first domain is said second domain.

5. The method of claim 1, wherein said second domain of said second oligonucleotide is disposed 5′ to said first domain of said second oligonucleotide.

6. The method of claim 1, wherein said first and second primers are not capable of amplifying DNA from another microorganism potentially present in said sample under appropriate conditions.

7. The method of claim 1, wherein said first type of microorganism is a bacterium.

8. The method of claim 7, wherein said bacterium is selected from the order Chlamydiales.

9. The methods of claim 1, wherein said first and second amplifications of DNA are amplifications by PCR.

10. The method of claim 1, wherein said sample comprises a biological material.

11. The method of claim 1, wherein, in step (c), amplified DNA of said DNA standard is purified to produce said first amplification product.

12. A pair of hybrid oligonucleotides comprising:
a) a first oligonucleotide comprising a first domain that binds to a first region of DNA of a first type of microorganism and is disposed at the 3′ end of said first oligonucleotide, and a second domain that binds to a first region of DNA of a second type of microorganism and is disposed 5′ to said first domain of said first oligonucleotide; and
b) a second oligonucleotide comprising a first domain that binds to a second region of DNA of said first type of microorganism and is disposed at the 3′ end of said second oligonucleotide, and a second domain that binds to a second region of DNA of said second type of microorganism and is disposed 5′ to said first domain,
wherein said first and second regions of DNA of said first type of microorganism flank a gene, or portion thereof, of said first type of microorganism, and said first and second regions of DNA of said second type of microorganism flank a gene, or portion thereof, of said second type of microorganism, and wherein said first and second oligonucleotides are together capable of amplifying DNA of said gene of said first type of microorganism, or portion thereof, under appropriate conditions.

13. The pair of hybrid oligonucleotides of claim 12, wherein each of said first and said second microorganisms is a bacterium.

14. The pair of hybrid oligonucleotides of claim 13, wherein each of said bacteria is independently selected from the order Chlamydiales.

15. A hybrid oligonucleotide comprising a first domain and a second domain, wherein said first domain binds to a region of DNA of a first type of microorganism and is disposed at the 3′ end of said oligonucleotide, and said second domain binds to a region of DNA of a second type of microorganism and is disposed 5′ to said first domain.

16. The hybrid oligonucleotide of claim 15, wherein each of said first and said second types of microorganism is a bacterium.

17. The hybrid oligonucleotide of claim 16, wherein each of said bacteria is independently selected from the order Chlamydiales.

18. The hybrid oligonucleotide of claim 17, wherein said first and second regions of DNA are disposed in the gene for the major-outer-membrane protein.

19. The hybrid oligonucleotide of claim 15, comprising a sequence selected from the group consisting of:

| | |
|---|---|
| 5′-CGTTTGGTAGTTCATTTGCATTGCTGATCAGCTGCTATGGAGGA-3′; | (SEQ ID NO: 17) |
| 5′-CGTTTGGTAGTTCATTTGCATTTTGCATTGGAAGATCGGTAGAG-3′; | (SEQ ID NO: 18) |
| 5′-GCTGATCAGCTGCTATGGAGGACGTTTGGTAGTTCATTTGCATT-3′; | (SEQ ID NO: 19) |
| and | |
| 5′-TTGCATTGGAAGATCGGTAGAGCGTTTGGTAGTTCATTTGCATT-3′. | (SEQ ID NO: 20) |
